(19) United States
(12) Patent Application Publication
(10) Pub. No.: US 2026/0110696 A1
(43) Pub. Date: Apr. 23, 2026

(54) SYSTEM AND METHOD OF IDENTIFICATION OF A SPECIFIC ALKYLAMIDE FOR THE TREATMENT OF RESPIRATORY VIRUS INFECTIONS

(71) Applicant: Sonoran University of Health Sciences, Tempe, AZ (US)

(72) Inventors: Jeffrey Langland, Chandler, AZ (US); Johanne Gerstel, Tempe, AZ (US); Keely Puchalski, Chandler, AZ (US)

(73) Assignee: Sonoran University of Health Sciences, Tempe, AZ (US)

(21) Appl. No.: 19/361,644

(22) Filed: Oct. 17, 2025

Related U.S. Application Data
(60) Provisional application No. 63/708,547, filed on Oct. 17, 2024.

(51) Int. Cl. G01N 33/68; A61K 31/16; G01N 33/50; G01N 33/543

(52) U.S. Cl. CPC G01N 33/6869; A61K 31/16; G01N 33/5023; G01N 33/5038; G01N 33/54366; G01N 2333/095; G01N 2333/11; G01N 2333/5421; G01N 2500/10

(57) ABSTRACT

A method is provided for reducing cytokine secretion in virus-infected cells through treatment with specific alkylamides. The method includes identifying a cell infected with a rhinovirus or influenza virus and administering one or more alkylamides, such as Dodeca-2(E),4(E)-Dienoic acid isobutylamide, to reduce cytokine release, particularly interleukin-8 (IL-8). In certain embodiments, the alkylamides are in purified form and may be derived from Echinacea purpurea. The method may further comprise measuring IL-8 secretion to assess efficacy of the treatment. Another aspect provides a screening method for identifying specific alkylamides effective against respiratory virus infections by comparing cytokine secretion levels between infected cells treated with purified alkylamides and those treated with Echinacea purpurea ethanolic extract. Alkylamides demonstrating reduced cytokine secretion and a selectivity index greater than 10 are identified as therapeutic candidates for mitigating cytokine-mediated inflammatory responses in respiratory viral infections.

### CROSS-REFERENCE TO RELATED APPLICATIONS

This application claims the benefit of priority of U.S. Application No. 63/708,547 entitled “System and Method of Identification of a Specific Alkylamide for the Treatment of Respiratory Virus Infections” to Jeffrey Langland et al., filed on October 17, 2024, the contents of which is hereby incorporated by reference in its entirety.

### BACKGROUND

### 1. Field of the Invention

Generally, the invention relates to.

### 2. Description of Related Art

The common cold is the most common illness known and leads to frequent symptoms including sneezing, raspy throat and nasal congestion. People in the U.S. have an estimated one billion colds each year and account for more visits to the doctor than any other condition. Rhinovirus, the most common virus associated with the cold, can lead to additional complications including sinus or ear infections and acute bronchitis. In addition, patients with asthma, chronic bronchitis, or emphysema, may have increased symptoms while infected.

Influenza (“flu”) is another common respiratory virus infection. On average, about eight percent of the U.S. population gets sick from flu each season. Symptoms following influenza infection are typically more severe than a rhinovirus infection and complications can include bacterial pneumonia, ear infections, sinus infections and worsening of chronic medical conditions, such as congestive heart failure, asthma, or diabetes.

Cytokines are proteins that are small signaling molecules produced by cells in the body that serve as molecular messengers between cells and play crucial roles in cell communication and immune responses. As part of the immune system, cytokines regulate inflammation, immune cell activation, and other immune-related processes and generally regulate the body’s response to disease and infection. When the immune system detects a threat, such as an infection, certain immune cells release cytokines to trigger a coordinated response to eliminate the threat. The specific cytokine, IL-8, is the primary cytokine involved in the recruitment of neutrophils to the site of an infection. This recruitment of immune cells is largely responsible for the symptoms associated with cold or flu infections.

Echinacea is a genus of flowering plants in the daisy family (Asteraceae) that is commonly used as an herbal remedy. Echinacea is particularly known for its potential immune-modulating properties and various parts of the Echinacea plant are used in traditional medicine and commercial supplements. One of the active classes of compounds found in Echinacea are alkylamides, also known as alkamides or alkylalkamides. These are a group of bioactive constituents primarily found in the roots and aerial parts (flowers and leaves) of the Echinacea plants. Alkylamides are thought to contribute to the medicinal effects of Echinacea, especially in terms of immune system support. Prior research suggests that alkylamides can influence the immune response by affecting cytokine production and activity.

### SUMMARY

Although previous research suggests that the alkylamides present in Echinacea may be responsible for reducing symptoms associated with the common cold or flu, the role of which specific alkylamides and their target (ie. immune and/or antiviral) has not been well elucidated or established. This invention tested the antiviral and cytokine regulatory activity of various pure alkylamides and found that one specific alkyamide, Dodeca-2E,4E-Dienoic acid isobutylamide, has potent antiviral activity against rhinovirus (the causative agent of most common colds) and influenza virus, and potent inhibition of IL-8 cytokine production, which is responsible for many symptoms associated with these viral infections. The broad activity and low cytotoxicity of this specific alkylamide supports its potential use for the treatment of rhinovirus and influenza virus infections.

Implementations of a method of reducing cytokine secretion of a cell may comprise identifying a cell that is infected with at least one of a rhinovirus or an influenza virus and treating the cell with one or more alkylamides, the one or more alkylamides comprising Dodeca-2(E),4(E)-Dienoic acid isobutylamide.

Particular aspects may comprise one or more of the following features. The cytokine may be interleukin-8 (IL-8). The one or more alkylamides may be in purified form. The one ore more alkylamides may be derived from Echinacea purpurea. The method may further comprise measuring a level of IL-8 secretion by the cell and determining a level of efficacy of the one or more alkyamides at reducing IL-8 secretion of a cell based on the measured level of IL-8 secreted by the cell in the presence of the one or more alkylamides.

Implementations of a method of identifying a specific alyklamide for treatment of a respiratory virus infection may comprise infecting a first cell with a respiratory virus infection in the presence of one or more purified alkylamides, measuring a level of cytokine secretion of the first infected cell using Enzyme-Linked Immunosorbent Assay (ELISA), infecting a first cell with a respiratory virus infection in the presence of Echinacea purpurea root ethanolic (EtOH) extract, measuring a level of cytokine secretion of the second infected cell using ELISA, comparing the measured levels of cytokine secretion of the first and second infected cells, and identifying the one or more alkylamides as a treatment for a respiratory virus infection when the measured level of cytokine secretion of the first infected cell is less than that measured level of cytokine secretion of the second infected cell.

Particular aspects may comprise one or more of the following features. The one or more purified alkylamides may comprise Dodeca-2(E),4(E)-Dienoic acid isobutylamide. The cytokine may be interleukin-8 (IL-8). The one ore more alkylamides may be derived from Echinacea purpurea. The one or more alkylamides identified as a treatment for a respiratory virus infection may have a selectivity index (SI) greater than 10.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventor is fully aware that he can be his own lexicographer if desired. The inventors expressly elect, as their own lexicographers, to use only the plain and ordinary meaning of terms in the specification and claims unless he clearly states otherwise and then further, expressly sets forth the “special” definition of that term and explains how it differs from the plain and ordinary meaning. Absent such clear statements of intent to apply a “special” definition, it is the inventors’ intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . “ or “step for performing the function of . . . ,” if the claims also recite any structure, material or acts in support of that means or step, or that perform the recited function, then it is the clear intention of the inventor not to invoke the provisions of 35 U.S.C. § 112(f). Moreover, even if the provisions of 35 U.S.C. § 112(f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

The foregoing and other aspects, features, and advantages will be apparent to those artisans of ordinary skill in the art from the DETAILED DESCRIPTION and DRAWINGS.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 shows graphical results of regulation of lipopolysaccharides (LPS) induced IL-8 secretion with Echinacea extract and alkylamides.

FIG. 2 is a graph showing results of rhinovirus plaque reduction assays with Echinacea extract and alkylamides.

FIG. 3 is a graph showing results of influenza plaque reduction assays with Echinacea extract and alkylamides.

FIG. 4 is a graphical depiction of CC50 and IC50 of effective alkylamide.

FIG. 5 is a chart providing a summary of activity of various alkylamides.

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below and it should be noted that while specific products and/or manufacturers may be referred to for exemplary purposes throughout this disclosure, other suitable substitutes are also contemplated.

Echinacea purpurea is a perennial medicinal herb with important immunostimulatory and anti-inflammatory properties, especially purpoted for the alleviation of cold and flu symptoms. Different classes of secondary metabolites of the plant such as alkamides, caffeic acid derivatives, polysaccharides, and glycoproteins are believed to be biologically and pharmacologically active. Although previous research suggests that the alkylamides present in Echinacea may be responsible for reducing symptoms associated with the common cold or flu, the role of which specific alkylamides and their target (ie. immune and/or antiviral) has not been well elucidated or established. This invention tested the antiviral and cytokine regulatory activity of various specific alkylamides present in Echinacea extracts and found that one specific alkyamide, Dodeca-2E,4E-Dienoic acid isobutylamide, has potent antiviral activity against rhinovirus (the causative agent of most common colds) and influenza virus, and potent inhibition of IL-8 cytokine production, which is responsible for most symptoms associated with the common respiratory infections. The broad activity and low cytotoxicity of this specific alkylamide supports its potential use for the treatment of rhinovirus and influenza virus infections.

### METHODS

### Botanical extract preparation:

For solvent extraction, dried Echinacea purpurea roots were ground to a fine powder, resuspended in extraction solution (70% Ethanol) and mixed continuously for 24 hours at room temperature. The extract was centrifuged at 3000xG for 10 min to remove plant debris and the extraction solution filtered through a 0.2 uM filter. The specific alklamides were obtained from Sigma-Aldrich® and prepared in dimethylsulfoxide (DMSO).

### Regulation of immune cytokine expression:

Differentiated THP-1 cells (human monocytes matured to macrophages by treatment with 10 uM phorbol 12-myristate 13-acetate (PMA)) were left untreated, treated with Echinacea extracts (50 ug/ml), or treated with alkylamides (1 ug/ml) followed by +/- treatment with lipopolysaccharide (1 ug/ml to induce cell activation and cytokine production) for 24 hours. The cell culture supernatant was collected and analyzed by enzyme-linked immunosorbent assay (ELISA) for IL-8. The expression level of IL-8 (pg/ml) was determined by comparison to a standard curve.

### Effect on virus replication:

For rhinovirus, HeLaH1 cells (ATCC CRL-1958) were maintained with Dulbecco's Minimum Essential Media supplemented with 10% heat inactivated fetal bovine serum and 1% Antibiotic-Antimycotic. Cells were maintained at 37°C, with 5% CO2 in a humidified chamber. Human rhinovirus 16 (ATCC VR-283) was propagated in HeLaH1 cells. HeLaH1 cells were infected with 100-200 plaque forming units (pfu) of rhinovirus in the presence of varying concentrations of Echinacea purpurea root EtOH extract or alkylamides for one hour at 37°C followed by incubation in media containing Echinacea extract or alkylamides for 3 days at 33°C. Plaques were visualized by staining with 0.1% crystal violet in 20% ethanol.

For influenza virus, Madin-Darby Canine Kidney (MDCK) cells (ATCC CCL-34) were maintained with Dulbecco's Minimum Essential Media (DMEM) supplemented with 10% heat inactivated fetal bovine serum and 1% Antibiotic-Antimycotic. Cells were maintained at 37°C, with 5% CO2 in a humidified chamber. Human influenza virus H1N1 (ATCC VR-1894) was propagated in MDCK cells. For viral assays, MDCK cells were washed twice with phosphate buffered saline (PBS). The virus was diluted in DMEM with 0.35% bovine serum albumin (BSA). Cells were infected with 100-200 pfu of diluted influenza virus in the presence of varying concentrations of Echinacea purpurea root EtOH extract or alkylamides for one hour at 37°C. The cell monolayer was washed twice with PBS followed by an overlay containing DMEM, 0.02% DEAE-Dextran, 0.03% MgSO4, 1 ug/ml TPCK-Trypsin, 0.6% agarose and comparable amounts of the Echinacea extract or alklyamides. The cells were incubated for 3 days at 37°C. Plaques were visualized by staining with 0.1% crystal violet in 20% ethanol.

### RESULTS

Interleukin-8 (IL-8) is a cytokine that plays a crucial role in the immune response to infections and inflammation. Rhinovirus is one of the most common viruses responsible for causing the common cold, and IL-8 is involved in the body's defense against this virus. When rhinovirus infects the respiratory tract, it triggers an immune response in the body. This response involves the release of various cytokines, including IL-8, by infected cells and immune cells. IL-8 acts as a chemoattractant, drawing immune cells to the site of infection to combat the virus. This increase in immune response is responsible for most of the symptoms associated with the common cold (and flu) including increased mucus production leading to nasal congestion or a runny nose, fatigue and headaches. Experiments were conducted to investigate the effect of alkylamides compared to Echinacea extract treatment on IL-8 synthesis in immune resting or stimulated cells. The Echinacea purpurea root was extracted in water or 65% EtOH. THP-1 cells were treated with the Echinacea extract or the various alkylamides followed by treatment or no treatment with an immune stimulant, lipopolysaccharide. The level of IL-8 production was measured by ELISA.

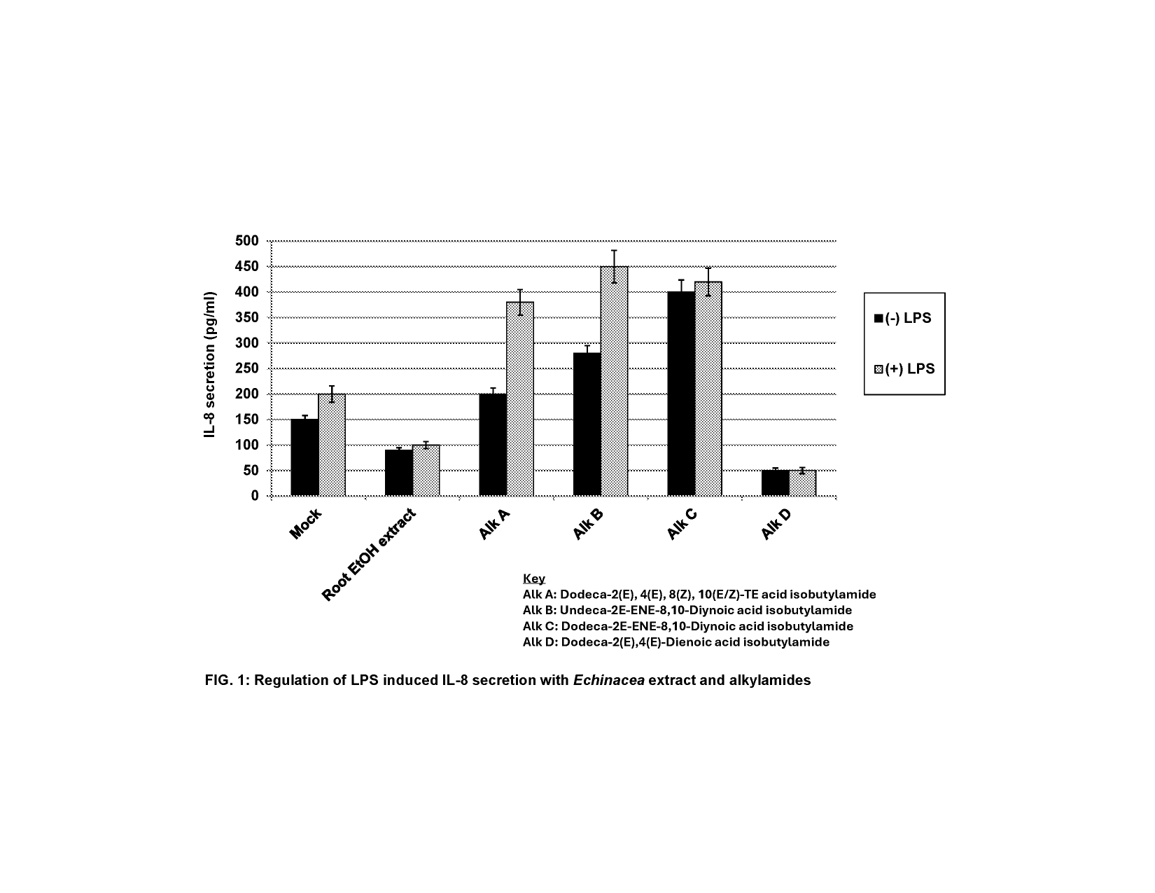

Lipopolysaccharides (LPS) are powerful activators of the immune response leading to the production of various cytokines, including IL-8. As shown in FIG. 1, treatment of cells with lipopolysaccharides (LPS) alone (Mock) led to an induction in IL-8 secretion as compared to no LPS treatment. Treatment with Echinacea root EtOH extract led to a reduction in IL-8 levels in both the presence or absence of LPS treatment. This supports previous results where Echinacea extracts are thought to induce anti-inflammatory repsonses. Since alkylamides are thought to be responsible for the immune modulatory effects of Echinacea, the cells were treated with various purified alkylamides in the presence and absence of LPS. As shown in FIG. 1, Alyklamides A, B and C (Dodeca-2(E), 4(E), 8(Z), 10(E/Z)-TE acid isobutylamide; Undeca-2E-ENE-8,10-Diynoic acid isobutylamide; and Dodeca-2E-ENE-8,10-Diynoic acid isobutylamide, respectively) all led to an INDUCTION in IL-8 levels, both in the absence or presence of LPS. Since the production of IL-8 during a common cold or flu infection leads to increased symptoms, it is proposed that if these alkylamides were used for the treatment of these infections, an undesirable increase in symptoms would likely be observed. When the cells were treated with Alkylamide D (Dodeca-2(E),4(E)-Dienoic acid isobutylamide), a DECREASE in IL-8 secretion was observed. This is a desireable effect similar to that observed with the crude Echinacea extract. Thus, treatment with this alkylamide during a common cold or flu infection would likely lead to reduced symptoms associated with the infection.

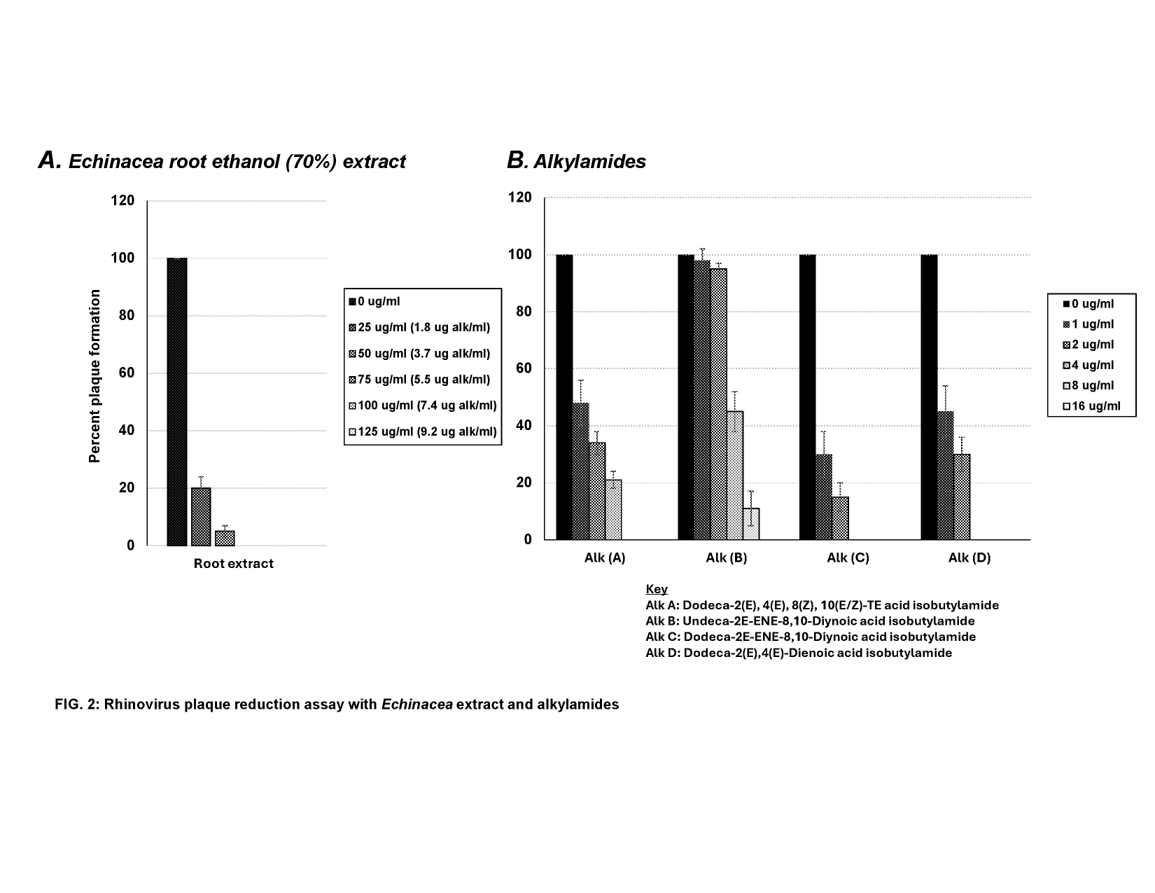

To examine the effects of an Echinacea purpurea extract and the various alkylamides on rhinovirus replication, HeLa H1 cells were infected with rhinovirus in the presence of increasing concentrations of the Echinacea extract (Part A) or the various alkylamides (Part B). As shown in FIG. 2, the Echinacea ethanol extract did reduce rhinovirus replication. When the various alkylamides were tested for anti-rhinovirus activity, all four alkylamides demonstrated some antiviral activity with Alkylamides C and D (Dodeca-2E-ENE-8,10-Diynoic acid isobutylamide and Dodeca-2(E),4(E)-Dienoic acid isobutylamide respectively) having the strongest activity. When the activity of these alkylamides was compared to the concentrations of alkylamides present in the crude Echinacea extract (Part A, alk/ml), comparible concentrations of total alkylamides present in the crude extract had similar levels of activity relative to the pure alkylamides (with a viral IC80 around 2 ug/ml for the crude extract and Alkylamides C and D). This supports that the alkylamides present in the crude Echinacea extract are likely responsible for the anti-rhinovirus activity, but that the level of antiviral activity does vary amongst the different alkylamides.

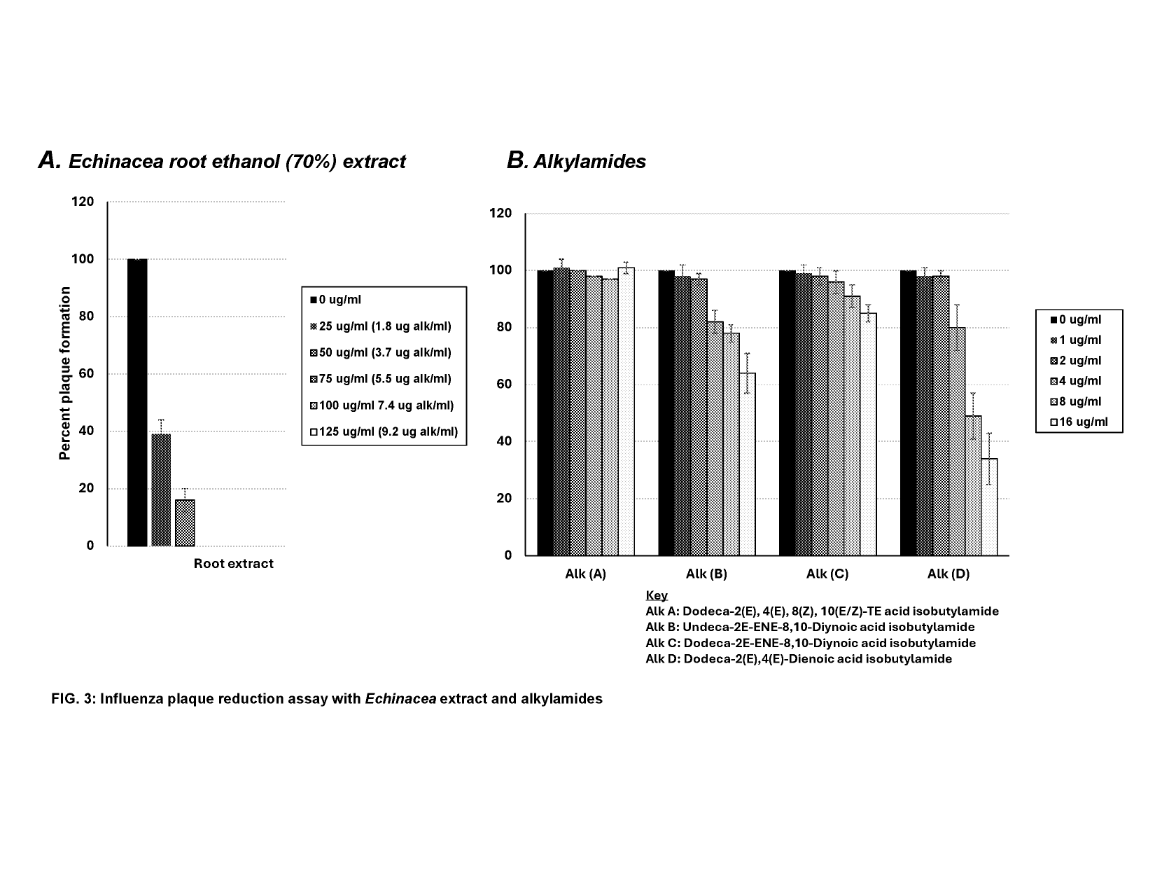

To examine the effects of the Echinacea purpurea extract and the various alkylamides on influenza virus replication, MDCK cells were infected with influenza H1N1 in the presence of increasing concentrations of Echinacea extract (Part A) or the various alkylamides (Part B). As shown in FIG. 3, the Echinacea purpurea extract effectively inhibited the replication of influenza virus. When the various alkylamides were tested for anti-influenza virus activity, Alkylamide D (Dodeca-2(E),4(E)-Dienoic acid isobutylamide) had the strongest antiviral activity with an IC50 of approximately 8 ug/ml. The other alkylamides demonstrated no or only minor anti-influenza virus activity. In comparison to the crude Echinacea extract, the total extract had more potent anti-influenza virus activity relative to the total alkylamide concentration, having an IC50 of approximately 1.5 ug total alkylamides/ml. This suggests that either a different alkylamide or a different secondary metabolite is responsible for the some of the anti-influenza virus activity observed in the crude extract.

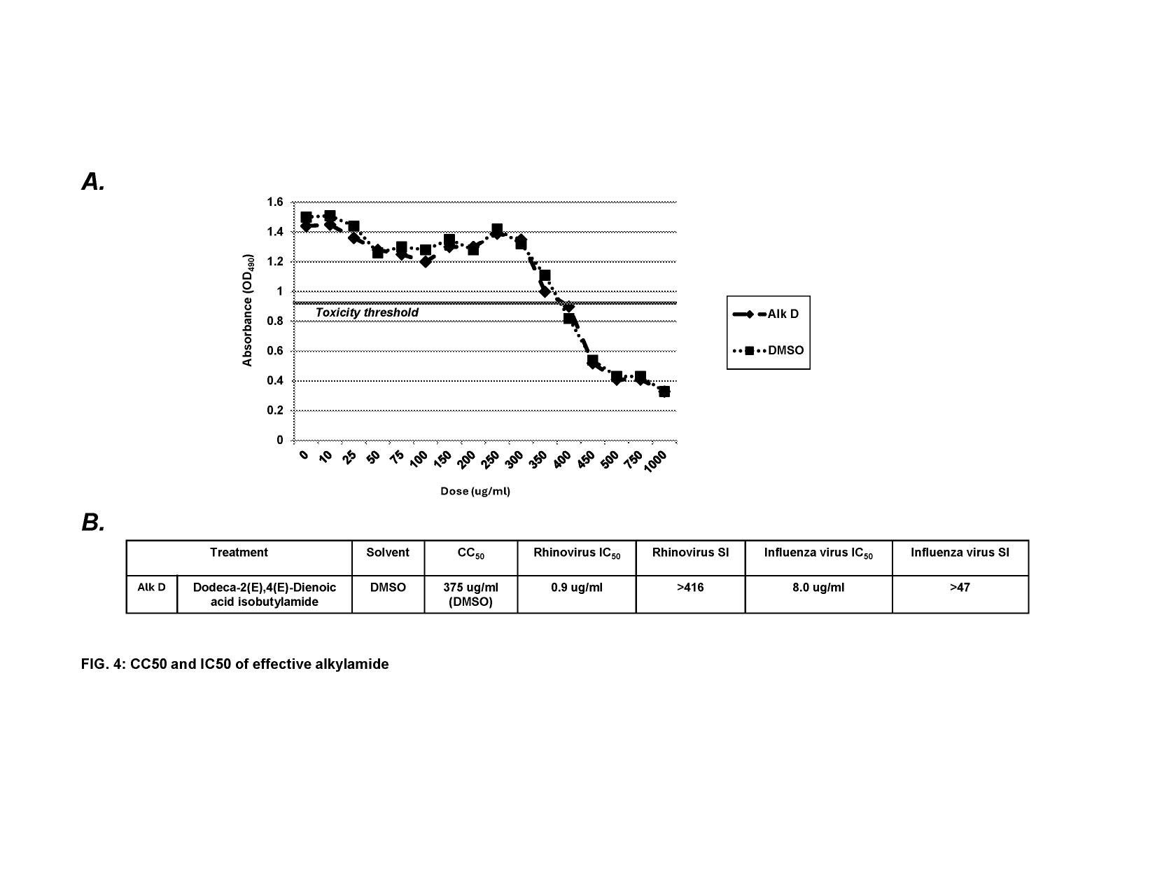

Since Alkylamide D (Dodeca-2(E),4(E)-Dienoic acid isobutylamide) had antiviral activity against both rhinovirus and influenza virus, and inhibited IL-8 secretion, this alkylamide has potential for use as a therapeutic for these infections. The selectivity index (SI) is a ratio between cytotoxicity and antiviral activity of a potential drug. The higher the SI ratio, the theoretically more effective and safe a therapeutic is during in vivo treatment for a given viral infection. Typically, relatively low of SI (< 1) means the compound could be toxic and should not be used as a therapeutic. If the calculated SI value is between 1 and 10, some concern is warranted and may be re-evaluated using other biosystems. Calculated SI values >10 are desirable and have more potential therapeutic value. As shown in FIG. 4, the SI for Alkylamide D (Dodeca-2(E),4(E)-Dienoic acid isobutylamide) is >47 for influenza virus and >416 for rhinovirus. These SI values are likely substantially higher since the CC50 for DMSO (the vehicle that the alkylamide is resuspended in) is the same as that for Alkylamide D. This suggests that the observed cell cytotoxicity is likely due to the DMSO and not the alkylamide. Therefore the SI for Alkylamide D (Dodeca-2(E),4(E)-Dienoic acid isobutylamide) is likely higher than that reported. These SI values are very high supporting the potential efficacy and safety for the use of Alkylamide D (Dodeca-2(E),4(E)-Dienoic acid isobutylamide) for the treatment of rhinovirus and influenza virus infections.

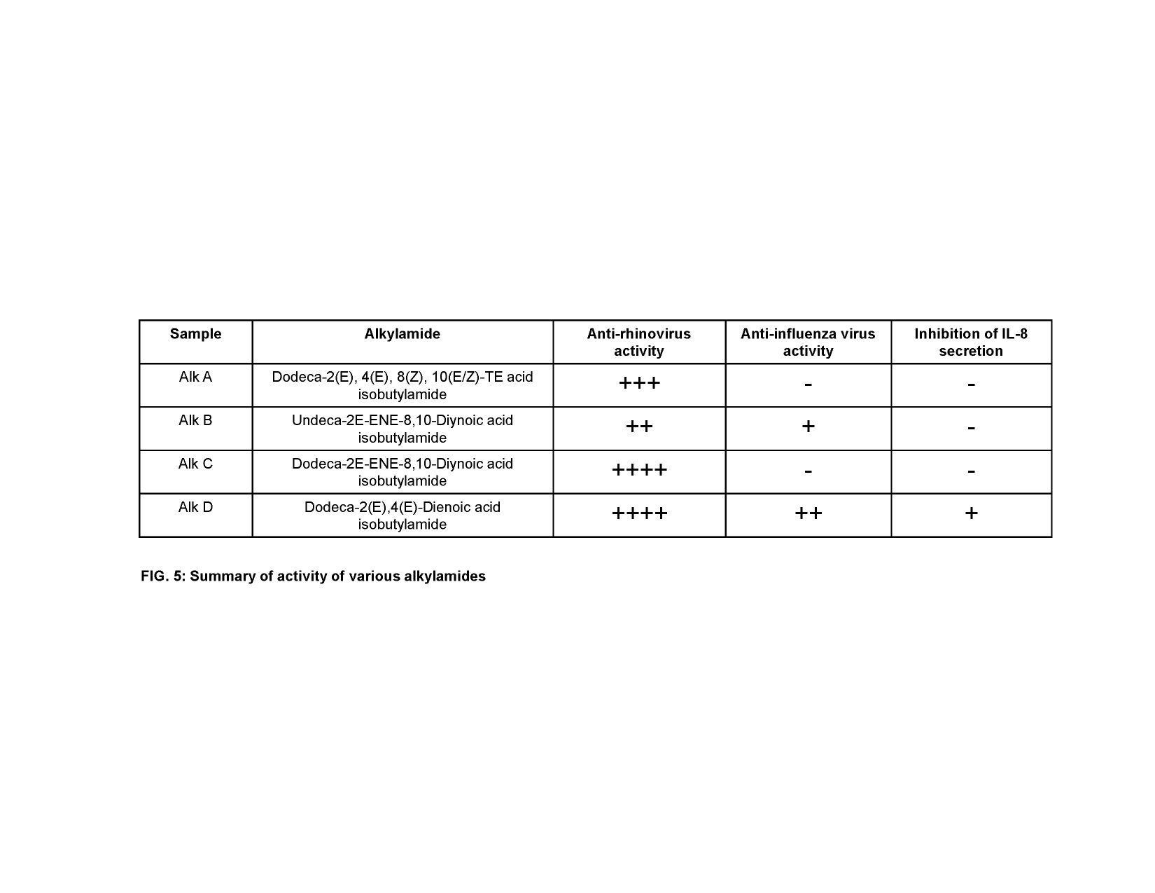

Alkylamides from Echinacea have been purported to be the most bioactive secondary metabolites present in the plant extracts. However, based on our studies, the activity of different alkylamides against viruses and immune modulation varies considerably. As summarized in FIG. 5, Alkylamides A, B, and C (Dodeca-2(E), 4(E), 8(Z), 10(E/Z)-TE acid isobutylamide; Undeca-2E-ENE-8,10-Diynoic acid isobutylamide; and Dodeca-2E-ENE-8,10-Diynoic acid isobutylamide, respectively) have antiviral activity against rhinovirus, but are ineffective against influenza virus and are actually pro-inflammatory leading to increases in IL-8 secretion. These specific alkylamides are likely not efficacious for the treatment of these viral infections. However, Alkylamide D (Dodeca-2(E),4(E)-Dienoic acid isobutylamide) demonstrated potent antiviral activity against both rhinovirus and influenza virus, and led to a decrease in IL-8 synthesis which, concomitantly is likely to lead to an improvement in symptoms in patients infected with either of these viruses.

In places where the description above refers to particular implementations of systems and methods for wound healing and infection reduction, it should be readily apparent that a number of modifications may be made without departing from the spirit thereof and that these implementations may be applied to other to systems and methods for wound healing and infection reduction.

1. A method of reducing cytokine secretion of a cell, the method comprising:
identifying a cell that is infected with at least one of a rhinovirus or an influenza virus;
treating the cell with one or more alkylamides, the one or more alkylamides comprising Dodeca-2(E),4(E)-Dienoic acid isobutylamide.

2. The method of claim 1, wherein the cytokine is interleukin-8 (IL-8).

3. The method of claim 1, wherein the one or more alkylamides are in purified form.

4. The method of claim 1, wherein the one ore more alkylamides are derived from Echinacea purpurea.

5. The method of claim 2, further comprising:
measuring a level of IL-8 secretion by the cell; and
determining a level of efficacy of the one or more alkyamides at reducing IL-8 secretion of a cell based on the measured level of IL-8 secreted by the cell in the presence of the one or more alkylamides.

6. A method of identifying a specific alyklamide for treatment of a respiratory virus infection, the method comprising:
infecting a first cell with a respiratory virus infection in the presence of one or more purified alkylamides;
measuring a level of cytokine secretion of the first infected cell using Enzyme-Linked Immunosorbent Assay (ELISA);
infecting a first cell with a respiratory virus infection in the presence of Echinacea purpurea root ethanolic (EtOH) extract;
measuring a level of cytokine secretion of the second infected cell using ELISA;
comparing the measured levels of cytokine secretion of the first and second infected cells; and
identifying the one or more alkylamides as a treatment for a respiratory virus infection when the measured level of cytokine secretion of the first infected cell is less than that measured level of cytokine secretion of the second infected cell.

7. The method of claim 6, wherein the one or more purified alkylamides comprises Dodeca-2(E),4(E)-Dienoic acid isobutylamide.

8. The method of claim 6, wherein the cytokine is interleukin-8 (IL-8).

9. The method of claim 6, wherein the one ore more alkylamides are derived from Echinacea purpurea.

10. The method of claim 6, wherein the one or more alkylamides identified as a treatment for a respiratory virus infection has a selectivity index (SI) greater than 10.
